(19) United States
(12) Patent Application Publication
(10) Pub. No.: US 2005/0181021 A1
(43) Pub. Date: Aug. 18, 2005

(54) Vitamin E phosphate/phosphatidycholine liposomes to protect from or ameliorate cell damage

(76) Inventor: Robert Lamb, Midlothian, VA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/070,738

(22) Filed: Mar. 2, 2005

Related U.S. Application Data
(62) Division of application No. 09/670,346, filed on Sep. 27, 2000, now abandoned.
Application No. 09/670,346 is a continuation-in-part of application No. 09/368,173, filed on Aug. 5, 1999, now abandoned.
Application No. 09/368,173 is a continuation-in-part of application No. 08/347,167, filed on Nov. 23, 1994, now abandoned.
Application No. 08/347,167 is a continuation of application No. 08/109,486, filed on Aug. 20, 1993, now abandoned.
Application No. 08/109,486 is a continuation-in-part of application No. 07/836,085, filed on Feb. 14, 1992, now abandoned.

(51) Int. Cl. A61K 31/665; A61K 47/00; A61K 9/127

(52) U.S. Cl. 424/439; 424/450; 514/100

(57) ABSTRACT

This invention relates to protecting cells from damage and stimulating cell repair by administration of vitamin E phosphate encapsulated in phosphatidylcholine liposomes.

### FIELD OF THE INVENTION

This invention relates to protecting cells from damage and stimulating cell repair by administration of vitamin E phosphate encapsulated in phosphatidylcholine liposomes.

### BACKGROUND OF THE INVENTION

A number of publications have discussed the merits of vitamin E in prevention of cell damage due to oxidative stress such as that caused by toxic injury. The protective properties of vitamin E have been attributed to its role as a membrane-active antioxidant. It is believed that vitamin E, a lipid soluble vitamin, dissolves in the phospholipid environment of the membranes and donates a hydrogen to terminate the free radical-induced peroxidation of the unsaturated fatty acids of membrane phospholipids. It has been generally accepted that it is by this mechanism that vitamin E protects cells from free radical-induced injury.

There is no question that vitamin E is an essential dietary requirement, since vitamin E deficiency results in structural and functional alterations in various tissues such as liver, brain, heart, muscle, etc. As a result, vitamin E has been used to treat various disorders of the heart, brain, liver and muscle. Unfortunately, vitamin E therapy has produced little or no benefit in most instances. This was not surprising, since results in cultures of hepatocytes suggest that vitamin E and vitamin E acetate (VEA) were relatively inactive. Hence, it was seen that the administration of vitamin E as a medicinal was of minimal benefit.

The use of vitamin E to protect specifically against chemical-induced toxicity has been known. (Burton, et al, “Vitamin E as an antioxidant in vitro and in vivo”, Biology of vitamin E, Pitman, London (1983) London. Also see Yoshikawa and Kondo, “Role of Vitamin E in the Prevention of Hepatocellular Damage—”, Vitamin E: Biochemical, Hematological, and Clinical Aspects, Lubin and Machlin, ed.; N.Y. Academy of Sci., (1982) 198-200.) Yoshikawa found no correlation between serum level of vitamin E and liver function, but did find a correlation between βlipoprotein, a carrier of vitamin E, and liver function. Disturbance of liver function appears to arise, in such instances, from failure of effective delivery of vitamin E to the cell rather than as a result of host deficiency of vitamin E.

It has also been known that even when protection from cell injury is demonstrated using vitamin E in cell culture, a similar response often is not seen in the intact animal. The laboratory of Dr. Reed at Oregon State University has directed attention to the mechanism of protection against chemical-induced toxicity using vitamin E succinate. (See Pascoe, et al., Archives of Biochemistry and Biophysics, Vol. 253, No. 1, pp 150-158 and pp. 159-166 (1987).)

The need for a method of protecting liver cells from toxicity is particularly important because many medications are metabolized to toxic metabolites in the liver. A method which effectively protects the liver from medicinal-induced toxic injury would permit the use of medications that are toxic to liver tissue. An example of a compound that could be used to alleviate a disease condition but is toxic to liver tissue is tetrahydroaminoacridine (THA), a compound that has shown promise for use in treatment of Alzheimer's disease, but which is not currently in use because it has proven to be too hepatotoxic. It has been shown that vitamin E and vitamin E succinate are useful in protecting the liver from chemical-dependent damage in vitro. However, as discussed previously, vitamin E has been found to be less useful in vivo in providing protection of the liver. (See Dogterom, et al, Biochemical Pharmacology. Vol 37, No 12, pp. 3211-2313 (1988).)

Attempts have been made to improve in vivo response by esterification of vitamin E. The most commonly used vitamin E esters are the acetate (VEA) and the succinate (VES) esters. (Fariss, et al, Toxicology Letters, 47 (1989) 61-75). Fariss' findings indicate that vitamin E succinate is superior to vitamin E and VEA in providing protection of cells from toxicant injury. However, the degree of protection seen in the cell cultures has not been reflected in protection of tissues in the intact animal.

The delivery of active agents to the site where beneficial effect is needed presents several problems. Many biologically active agents are quickly destroyed in the body by enzymes before they reach their intended target tissue. Furthermore, some drugs are unable to cross membrane barriers. The ‘packaging’ of pharmaceutically active agents to avoid destruction in the body's environment and to effectively deliver active agents across membrane barriers has, for many years, been accomplished by the use of liposomes, microdroplets, and microcrystals. Liposomes consist of phospholipid vesicles containing water-soluble drugs (See, for example, U.S. Pat. No. 4,241,046, which is incorporated herein by reference). They consist of a spherical lipid bilayer with an aqueous phase inside. Other preparations such as microdroplets (See U.S. Pat. No. 4,725,442, which is incorporated herein by reference) and microcrystals (See Patent Publication WO 91/16068) have also been used. The vitamin E phosphate, as disclosed herein, may advantageously be administered in any of the above formulations.

The need for medicinals that will reduce alcohol-induced liver injury and stimulate liver cell repair is urgent, especially among women and persons of color, who respond to ingestion of alcohol with much higher levels of cirrhosis of the liver. The use of vitamin E in a form that would be effective in preventing cell damage and repairing damage to liver cells from exposure to ethanol in a form that would not be destroyed in the serum has not previously been known. The delivery of the vitamin E phosphate using phosphatidylcholine liposomes is effective in reversing the deleterious effects of alcohol on liver cells.

### SUMMARY OF THE INVENTION

It is the purpose of this invention to provide means for protecting cells from (prophylaxing against) damage and for providing means for reversing cell damage by administration of vitamin E phosphate in the form of liposomes, particularly those prepared with phosphatidylcholine.

### DISCLOSURE OF THE INVENTION

It has now been found that vitamin E phosphate protects cells from effects of oxidative stress and enhances the repairing process in damaged cells such as that which is chemically-induced. The vitamin E phosphate in phosphatidylcholine liposomes is particularly useful for protecting the tissue or ameliorating cell damage in the intact animal. A preferred route of administration for effecting protection of liver tissue is intraperitoneal injection or infusion. The carrier used in the vitamin E phosphate/phosphatidylcholine liposome-containing compositions and the mode of administration will depend on the target organ. The phosphatidylcholine both protects the vitamin E phosphate from inactivation in the serum and enhances the cellular repairing properties of the composition. The vitamin E phosphate/phosphatidylcholine liposomes provide benefits not available when administering the two components separately, even though they may be administered simultaneously.

Because the growth of liver cells in tissue culture is very useful for research, for diagnostic purposes and for production of products of the liver in vitro, the use of the vitamin E phosphate/phosphatidylcholine in tissue culture is also an important embodiment of this invention.

Materials and Methods:

The vitamin E phosphate was obtained from Sigma.

Waymouth Media disclosed below was used to grow hepatocytes:

Waymouth 752/1 amino acids (without valine) (A.A. Mix)

| Amino Acid | gm/50 liters |
|---|---|
| L-Aspartic Acid | 3.0 gm |
| L-Cystine | 0.75 gm |
| L-Glutamic Acid | 7.5 gm |
| Glycine | 2.5 gm |
| L-Isoleucine | 1.25 gm |
| L-Leucine | 2.5 gm |
| L-Proline | 2.5 gm |
| L-Threonine | 3.75 gm |
| L-Tyrosine | 2.0 gm |
| Cysteine | 3.05 gm |
| L-Histidine | 6.4 gm |
| L-Lysine | 12.0 gm |
| L-Tryptophan | 2.0 gm |
| L-Methionine | 2.5 gm |
| L-Phenylalanine | 2.5 gm |
| L-Ornithine | 1.0 gm |

The amino acids were mixed well with mortar and pestle. Mixture was stored at room temperature in a dark bottle.

Monolayer Supplement (#1)

| | mg/ml | | mg/ml |
|---|---|---|---|
| Glucagon | 0.71 | B-Estradiol | 0.75 |
| Testosterone | 0.79 | Dexamethasone | 0.79 |
| Oleate | 5 | Linoleate | 5 |

This mixture of hormones and fatty acids is made and stored frozen in 0.5 ml aliquots.
Monolayer Supplement #2

A mixture of aminolevulinic acid, 1.7 mg/ml and selenium, 0.017 mg/ml is stored frozen in 0.1 ml aliquots.

Waymouth 752/1 media: Preparation of 10 liters

| | | | | | |
|---|---|---|---|---|---|
| H₂O | 7 | liters | AA mix | 11.15 | g |
| HCl | 1.515 | g | KH²PO₄ | 0.808 | g |
| MgSO₄ | 0.9899 | g | NaCl | 69.39 | g |
| Na₂HPO₄ | 8.989 | g | CaCl₂.H₂O | 1.212 | g |
| MgCl₂.6H₂O | 2.424 | g | NaHCO₃ | 7.07 | g |

Adjust pH to 7.40, adjust volume to 10 L. having water osmolarity of 280-295.
Hepatocyte Culture Medium:

To 500 ml. Waymouth 752/1 media add:
0.5 ml aliquot of monolayer supplement #1
0.1 ml aliquot monolayer supplement #2
5 ml Glutamine stock solution (17.5 g/500 ml)
5 ml glucose stock solution (180 g/500 ml)
5 ml valine stock solution (3.25 g/500 ml, pH 7.4)
5 ml Penicillin stock solution (5,000,000 units/500 ml)
5 ml gentamicin stock solution (5 mg/ml)
5 ml Vitamin C stock solution (5 mg/ml)
1.25 ml insulin (100 units/ml.)
10 ml pyruvic acid stock solution (11.22 g/100 ml)

### EXAMPLE 1

Liposomes containing the calcium salt were prepared. Phosphatidylcholine (200 mg) was dissolved in 5 ml of DMSO. 200 mg of the vitamin E phosphate (calcium salt) was added. The mixture was sonicated 5 min. at 37° C. degrees. Fifteen ml of 0.9% saline was added. The mixture was then sonicated for 15-30 minutes at 37° C.

### EXAMPLE 2

Influence of vitamin E phosphate/phosphatidylcholine liposomes (see example 1) on Allyl alcohol-induced liver injury was evaluated in male albino mice.

| | Treatment |
|---|---|
| SGPT | % of control ± SEM |
| Control (9) vehicle only | 100 ± 7 |
| Allyl alcohol (9) | 330 ± 15** |
| Vehicle plus VEP/PC (9) | 100 ± 3** |
| Allyl alcohol plus VEP/PC (9) | 109 + 4** |

VEP/PC is vitamin E phosphate/phosphatidylcholine
**The mice were exposed to a single intraperitoneal dose of allyl alcohol (50 mg/kg) or vehicle for 4 hours.

### EXAMPLE 3

Compositions using the sodium salt of the vitamin E phosphate were prepared in the following manner:

To 1 part (10 mg) of vitamin E phosphate sodium salt was added 4 parts (40 mg) of phosphatidylcholine. There was added sufficient sterile water to yield a total volume of 5 ml. The composition was then sonicated at 37° C. for 10 to 15 minutes. The preparation was then sterilized by irradiation. The liposomes formed using the sodium salt proved to be more preferred than either the microcrystals or the liposomes prepared using the calcium salt of the vitamin E phosphate.

The use of vitamin E as disclosed in the prior art as an agent to protect cells from toxic injury has shown little or no promise for use as a therapeutic in vivo. It is now seen that the phosphate ester of the vitamin, when formulated in a manner that prevents hydrolysis by esterases in the gut and serum, can be used to protect cells from toxic injury in vivo. When treating the intact animal, any technology that delivers the phosphate ester of the vitamin E to the tissues subject to damage from oxidative stress such as, for example, exposure to toxins, including those occasioned by therapeutic agents, will be appropriate. The use of liposome technologies to protect the active agents provides a useful means of delivery.

A particularly useful function of vitamin E phosphate/phosphatidylcholine liposomal compositions of the present invention is to prevent or treat liver cell damage caused by the administration of a therapeutic agent to a patient. As indicated above, a number of therapeutic agents are known to be the cause of liver damage, such as, for example, the THA previously mentioned, as well as a number of anti-cancer drugs. The liver cell damage can be counteracted by administering a protective amount of vitamin E phosphate/phosphatidylcholine liposomes at a time which is proximate in time to the administration of the therapeutic agent to the patient. It may be convenient to administer the liposomes concurrently with the therapeutic agent, but it will be readily appreciated by those in the art that the time of administration of the liposomes of the present invention can be prior in time or subsequent in time to the administration of the therapeutic agent. Normally, the liposomes will be administered to the patient within 30 minutes of the time of administration of the therapeutic agent, but that time can vary depending upon how rapidly the liver cells in the patient are contacted by the therapeutic agent or liver cell toxic metabolite thereof, and the time of administration of the liposomes, relative to the time of administration of the therapeutic agent, can be readily determined by those in the art.

### EXAMPLE 4

The vitamin E phosphate/phosphatidylcholine liposomes were administered at the level of 25 μM in cell growth media to evaluate comparative effectiveness against differing kinds of cells. The effect was evaluated by measuring alterations in the incorporation of ³H-choline into phosphatidylcholine of various cells incubated for 4 hours and 72 hours.

Incubation Time

| | |
|---|---|
| 4 hours | 72 hours |

| | |
|---|---|
| Type of Cells | Percent of control ± SEM |

| | | |
|---|---|---|
| Rat liver | 138 ± 8 | 860 ± 49* |
| Mouse macrophages | 121 ± 4 | 396 ± 49* |
| Mouse Intestine | 493 ± 18* | 517 ± 20* |

*indicates level of significance from control (-vitamin E phosphate) is p ≦ 0.01.

The data demonstrates that vitamin E phosphate stimulates membrane repair processes (phosphatidylcholine biosynthesis) in various cells. It has been found that the vitamin E phosphate, in the presence of divalent metal salts, is precipitated. Liposomes were made using salts of the vitamin E phosphate and phosphatidylcholine. These salts were protected by liposomes to avoid precipitation in the presence of 2⁺ metal ions.

Considering example 2, it can be seen that the administration of vitamin E phosphate/phosphatidylcholine in conjunction with allyl alcohol protected cells from damage. The following example demonstrates the ability of vitamin E phosphate/phosphatidylcholine to stimulate repair of previously damaged cells.

### EXAMPLE 5

Liver cells which had become dysfunctional due to exposure to ethanol were incubated 24 hours with vitamin E phosphate/phosphatidylcholine liposomes. The pmoles of phosphatidylcholine formed per minute/mg protein was then measured with the following results:

| Control | ETOH* | ETOH/water** | ETOH/VEP/PC*** |
|---|---|---|---|
| 75 | 25 | 27 | 85 |

wherein
VEP/PC represents vitamin E phosphate/phosphatidylcholine
*represents the cells which were exposed to ethanol
**represents the cells which were exposed to ethanol, then incubated for 24 hours in water
***represents cells which were exposed to ethanol, then incubated for 24 hours in the presence of vitamin E phosphate in phosphatidylcholine liposomes.

A modified technique makes it possible to make liposomes inexpensively. The resulting liposomes may be used in beverages as a supplement to the diet.

### EXAMPLE 6

A composition was prepared by adding to 140 milligrams of sodium salt of vitamin E phosphate in 40 ml of deionized water to 400 mg of soybean phosphatidylcholine (polyenylphosphatidylcholine). The mixture was sonicated until a clear solution was formed (5 to 10 minutes). The resulting vitamin E phosphate/phosphatidylcholine liposomes were sterilized by filtration through 0.22 μm Millipore filters. The resulting vitamin E phosphate/phosphatidylcholine liposomes were found to be stable for several months. The use of polyenylphosphatidylcholine in liposomes containing vitamin E phosphate provides a particularly useful product. It has now been found that the combination provides benefits which are illustrated by further examples below. Hence, use of polyenylphosphatidylcholine, which is obtained from soybeans, provides a preferred form of phosphatidylcholine for use in compositions and methods of the invention. Hence, use of polyenylphosphatidylcholine provides a preferred form of vitamin E phosphate/phosphatidylcholine.

### EXAMPLE 7

The disodium salt of vitamin E phosphate was prepared several times using the following procedure. The dl-alpha-tocopherol, 25 grams (40 mmoles) (95% purity from Sigma) was dissolved in 75 ml toluene in a flat bottom 500 ml flask fitted with a cooling condenser. Ten grams (10 ml) [126.6 mmoles] of pyridine was added. The mixture was cooled in an ice bath. After cooling, 12.3 grams (7.5 ml) [80 mmoles] phosphorus oxychloride was added very slowly (dropwise) with mixing. The mixture was removed from the ice bath and stirred for 3 hours at room temperature. The reaction mixture was again cooled and 100 ml of water was added slowly with stirring. The mixture was then removed from the ice bath and placed in a cooling condenser flask, the refluxed at 90° C. for 4 hours with stirring.

The mixture was then transferred to a sepratory funnel. The flask was washed with toluene and wash material was added to mixture in the sepratory funnel. The contents of the funnel were allowed to rest over night, after which the phases were separated. (Both upper and lower phases will be clear when separation is complete.) The (bottom) layer was removed and the organic phase transferred to a rotoevaporator.

The organic phase was concentrated to dryness. The resulting solid was dissolved in 30 ml isopropyl alcohol with heat and stirring, then filtered. Thereafter, 50 ml of methyl alcohol containing 4.7 gm of sodium hydroxide was added slowly with vigorous mixing at room temperature until a precipitate was formed. The white precipitate was collected by filtration, then dissolved in 800 to 1000 ml methanol with heating. The solution was transferred to a rotoevaporator and reduced to 150 to 200 ml volume. Thereafter, 200 ml of acetone was added dropwise until a white precipitate was formed. The precipitate was collected by filtration, washed with acetone, then dried in a vacuum oven to provide, as a final product, the disodium salt of vitamin E phosphate. Yield was between 16 and 20 grams of the disodium salt of vitamin E phosphate.

### EXAMPLE 8

The following procedure was used in making the vitamin E phosphate/phosphatidylcholine liposomes. Phospholipon 90 G, a polyenylphosphatidylcholine, (purchased from American Lecithin Company in Connecticut) 35 grams, and 12.5 grams of vitamin E phosphate were placed in 750 ml of deionized water in a blender, then mixed at low speed for 1-2 minutes. (It is important not to allow foaming to occur.) The resulting mixture was then added to 2 liters of deionized water in a 4 liter glass or stainless steel beaker. (Plastic beakers will allow the solution to become too hot during sonication.) The blender was washed with 750 ml of deionized water and the wash solution was added to the vitamin E phosphate and phosphatidylcholine solution in the beaker. The resulting volume of 3.5 L, is stirred for 2-3 hours with a magnetic stirrer.

Two L of the material from the stirred solution are placed in a 4 liter beaker in an ice bath. The sonicator horn (Branson 450 Sonifier with 1 inch diameter horn) was inserted into the solution. The solution was sonicated at maximum intensity for 30 minutes. The process converted the vitamin E phosphate/phosphatidylcholine mixture into a solution containing smaller and more uniform liposomal particles. The resulting product was then filtered to remove any non-solubilized material.

The above process provided a clear product. Prior products produced by other means did not provide a clear product. It is important that products used in beverages be clear in order to encourage consumer acceptance. The clear product can be added to beverages to provide benefits as disclosed herein. Examples of such beverages are tea-based beverages, beverages containing coffee, fruit juices, soy products, dairy products and carbonated beverages.

### EXAMPLE 9

The influence of phosphatidylcholine (100 μM), vitamin E phosphate (50 μm) and combination of vitamin E phosphate/phosphatidylcholine was compared. It was found that iron-dependent lipid peroxidation in isolated liver cells (1000×g cell fraction) is significantly reduced by phosphatidylcholine, vitamin E phosphate and vitamin E phosphate/phosphatidylcholine as measured by liver cell lipid peroxidation (LCLP). The study of effects on liver cells was also considered based on evaluation of effect on increase in the incorporation of labeled choline into phosphatidylcholine (phosphatidylcholine biosynthesis) by liver cells. Vitamin E phosphate/phosphatidylcholine produced greater beneficial effects than either vitamin E phosphate or phosphatidylcholine.

% control + SEM

| Additions | LCLP | PC biosynthesis |
|---|---|---|
| None | 100 ± 2 | 100 ± 3 |
| 1 mM iron | 357 ± 12* | 254 ± 9* |
| 1 mM iron + PC | 281 ± 4** | 167 ± 4** |
| 1 mM iron + VEP | 167 ± 3** | 145 ± 4** |
| 1 mM iron + VEP/PC | 124 ± 2** | 117 ± 4** |

wherein:
PC represents phosphatidylcholine
VEP represents vitamin E phosphate
VEP/PC represents vitamin E phosphate/phosphatidylcholine
*level of significance from control [none] is p < 0.05
**level of significance from iron is p < 0.05

### EXAMPLE 10

The use of vitamin E phosphate/phosphatidylcholine to effect reversal of damage from ethanol on liver cells was then studied. The cultured liver cells were incubated with 25 mM or 50 mM ethanol for 96 hours, at which time there is significant reduction in liver cell function (phosphatidylcholine biosynthesis). The effect of adding vitamin E phosphate/phosphatdylcholine after 72 hours was studied, and 24 hours later (96 hours after initial exposure to ethanol) values were obtained.

| Additions (exposure time) | % control ± SEM |
|---|---|
| None (96 h) | 101 ± 6 |
| 25 mM ethanol (96 h) | 38 ± 2* |
| 50 mM ethanol (96 h) | 22 ± 1* |
| 25 mM ethanol (96 h) + VEP/PC (24 h) | 101 ± 8** |
| 50 mM ethanol (96 h) + VEP/PC (24 h) | 68 ± 3** |

wherein:
VEP/PC represents vitamin E phosphate/phosphatidylcholine
*level of significance from control [none] is p < 0.05
**level of significance from ethanol exposed cells is p < 0.05

Vitamin E phosphate/phosphatidylcholine liposomes are particularly useful in cosmetic lotions and creams to ameliorate damage caused by oxidative stress and to increase repair functions of epithelial cells. Vitamin E phosphate/phosphatidylcholine liposomes may be added to foods or beverages to supplement the diet or given orally in tablet or capsular form to protect from the damaging effects of oxidative stress and to assist in cell repair functions. The vitamin E phosphate/phosphatidylcholine can also be used as a dietary supplement either alone or in conjunction with other dietary enhancing components.

Because many otherwise useful drugs are not given because of their effect on liver cells, the use of vitamin E phosphate/phosphatidylcholine liposomes given in conjunction with such drugs can provide useful benefits. Such drugs include, for example, tetrahydroaminoacridine (THA), which is useful in treatment of Alzheimer's, and several anticancer agents, such as tamoxifen, which cause liver damage. Administration with vitamin E phosphate/phosphatidylcholine to protect the liver may render such drugs far less objectionable as long-term treatments.

The vitamin E phosphate/phosphatidylcholine liposomes can, in accord with the teachings herein, be added to solutions used for storage and transport of tissues for transplant. One of the major problems in the transport of organs is the damage to cells between the time the organ is harvested and the time the organ is connected to the recipient's blood supply. The use of vitamin E phosphate/phosphatidylcholine liposomes to prevent tissue damage could greatly assist in improving the efficacy of such transplants. The concentration of the vitamin E phosphate/phosphatidylcholine liposomes can vary greatly. For example, concentrations of 1 μM to 1000 μM would be appropriate. A preferred concentration is 10 μM to 100 μM. The vitamin E phosphate in the vitamin E phosphate/phosphatidylcholine liposomes may be in the form of one of the soluble salts, such as the sodium or potassium salts, in isotonic solution. The use of vitamin E phosphate/phosphatidylcholine liposomes as an additive to such to solution for storage and transport would be useful with any tissue for transplant, such as heart, liver, muscle (including heart muscle), lung, kidney tissue.

The particular method used to deliver the vitamin E phosphate/phosphatidylcholine liposomes of the invention to the tissues of the intact animal will depend on the target tissue to which it is administered.

The vitamin E phosphate/phosphatidylcholine may be delivered to the heart muscle by any means that will deliver the vitamin E phosphate/phosphatidylcholine to the heart tissue, including by intravenous injection or by infusion into the heart muscle.

Compositions containing vitamin E phosphate/phosphatidylcholine liposomes may be delivered as mists or aerosols to the respiratory system or directly to tissues during surgery. They may be infused into tissues during or following transplant or surgery in an isotonic solution such as normal saline. The compositions of the invention can be delivered in drops (for example, as eye drops) or as infusions to the target tissues.

Compositions containing vitamin E phosphate/phosphatidylcholine liposomes may be administered to the skin as creams, gels, or liquids or may simply be added to cosmetic products. The protective vitamin E phosphate/phosphatidylcholine may be administered to minimize skin damage and could be added to cosmetics to prevent damage from exposure to toxins, radiation or burning. When the vitamin E phosphate/phosphatidylcholine is applied to tissue that has been burned or abraded, the application of the composition as a sterile isotonic solution may be appropriate.

The compositions of the invention can also be administered as suppositories or in depo agents.

Compositions of the invention can be administered intrathecally to facilitate contact of the active agent with neuronal tissue after head injury. However, the vitamin E phosphate/phosphatidylcholine may also be administered intravenously. During brain surgery, it could be administered directly to the brain tissue.

One theory that could account for the beneficial effect of vitamin E phosphate/phosphatidylcholine is that vitamin E phosphate is particularly capable of partitioning in the phospholipid bilayer of the membrane.

It is believed that the cellular toxicity of most drugs and chemicals is associated with “oxidative stress” and a rise in membrane-bound free radicals. Free radicals can alter the structure and function of cellular components by rapidly interacting with such components in the cell as unsaturated or sulfur-containing amino acids, nucleic acids, and unsaturated fatty acids of phospholipids. The vitamin E phosphate/phosphatidylcholine, which is able to resist destructive enzymes in the body and is able to partition the membranes of cells, is readily delivered to the cells in a form which counteracts the effect of free radicals, thus preventing damage and ameliorating untoward effects previously suffered by the cells. The vitamin E phosphate/phosphatidylcholine is capable both of prophylaxing against cell damage and stimulating repair of previously damaged cells.

For humans, sufficient vitamin E phosphate/phosphatidylcholine to deliver from 1 mg to 500 mg of vitamin E phosphate is appropriate. The phosphatidylcholine component of vitamin E phosphate/phosphatidylcholine liposomes is usually 2.5 to 5 times that of the vitamin E phosphate. Hence, the total amount of vitamin E phosphate/phosphatidylcholine will vary depending on the amount of phosphatidylcholine which is used in the particular formulation.

1. A beverage containing a product of sonication of vitamin E phosphate/phosphatidylcholine liposomes.

2. A method of preparing a beverage comprising:
(1) sonicating liposomes containing vitamin E phosphate and phosphatidylcholine until a clear liquid is obtained, then
(2) adding the clear liquid product of step 1 to a beverage.

3. A method of claim 2 wherein the phosphatidylcholine is polyenylphophatidylcholine.

4. The beverage of claim 1 which is a fruit drink.

5. The beverage of claim 1 which is a coffee or tea drink.

6. The beverage of claim 1 which is a soy product.

7. The beverage of claim 1 which is a dairy product.

8. The beverage of claim 1 which is a carbonated beverage.

9-24. (canceled)

25. A composition of matter comprising vitamin E phosphate/phosphatidylcholine liposomes in a food, beverage or nutritional supplement.

26. (canceled)
